(19) United States
(12) Patent Application Publication
(10) Pub. No.: US 2001/0048940 A1
(43) Pub. Date: Dec. 6, 2001

(54) CATIONIC AMPHIPHILE MICELLAR COMPLEXES

(76) Inventors: JENNIFER D. TOUSIGNANT, CAMBRIDGE, MA (US); SIMON J. EASTMAN, HUDSON, MA (US); EDWARD R. LEE, NATICK, MA (US); RONALD K. SCHEULE, HOPKINTON, MA (US); SENG H. CHENG, WELLESLEY, MA (US); J. NIETUPSKI, MILLBURY, MA (US); QIUMING CHU, MELROSE, MA (US); JOHN MARSHALL, HOPEDALE, MA (US)

Correspondence Address: GENZYME CORPORATION C/O FINNEGAN, HENDERSON,, FARABOW, GARRETT & DUNNER, FINNEGAN, HENDERSON, FARABOW, GARRETT, & DUNNER, LLP. 1300 I STREET, N.W., SUITE 700, WASHINGTON, DC, 20005-3315, US

(21) Appl. No.: 09/335,689

(22) Filed: Jun. 18, 1999

(51) Int. Cl. A61K 48/00; A61K 9/22; A61K 9/127

(52) U.S. Cl. 424/450; 424/468; 514/44; 435/458

(57) ABSTRACT

The effective introduction of foreign genes and other biologically active molecules into targeted mammalian cells is a challenge still facing those skilled in the art. Gene therapy, for example, requires successful transfection of target cells in a patient. The present invention relates to novel micellar complexes of cationic amphiphilic compounds that facilitate delivery of biologically active molecules to the targeted cells of a mammal. The novel micellar complexes are comprised of a cationic amphiphile, a biologically active molecule, a derivative of polyethylene glycol (PEG), and optionally, a co-lipid. A further aspect of the invention is the use of targeting agents in any of the methods that effectuate the delivery of biologically active molecules into the cells of mammals. A targeting agent is usually any molecule, peptide sequence, or large protein that preferentially targets or binds to specific mammalian cells.

The present invention relates to novel micellar complexes of cationic amphiphilic compounds that facilitate delivery (and/or transfection) of biologically active molecules to the targeted cells of a mammal. More particularly, the present invention relates to the unique properties of these micellar complexes and the methods of making and using micelles of cationic amphiphiles to enhance delivery of biologically active molecules to the desired cells of a mammal. A goal of the invention is to provide novel complexes that can be used in gene therapy. The invention also relates to the use of targeting agents that facilitate delivery of a biologically active molecule to a specific type of mammalian cell.

The effective introduction of foreign genes and other biologically active molecules into targeted mammalian cells is a challenge still facing those skilled in the art. Gene therapy requires successful transfection of target cells in a patient. Transfection, which is practically useful per se, may generally be defined as a process of introducing an expressible polynucleotide (for example a gene, a cDNA, or an mRNA) into a cell. Successful expression of the encoding polynucleotide thus transfected leads to production in the cells of a normal protein and is also practically useful per se. A goal, of course, is to obtain expression sufficient to lead to correction of the disease state associated with the abnormal gene.

Examples of diseases that are targets of gene therapy include: inherited disorders such as cystic fibrosis, Gaucher's disease, Fabry's disease, and muscular dystrophy. Representative of acquired target disorders are: (1) for cancers—multiple myeloma, leukemias, melanomas, ovarian carcinoma and small cell lung cancer; (2) for cardiovascular conditions—progressive heart failure, restenosis, and hemophilias; and (3) for neurological conditions—traumatic brain injury.

Cystic fibrosis, a common lethal genetic disorder, is a particular example of a disease that is a target for gene therapy. The disease is caused by the presence of one or more mutations in the gene that encodes a protein known as cystic fibrosis transmembrane conductance regulator (“CFTR”). Cystic fibrosis is characterized by chronic sputum production, recurrent infections and lung destruction (Boat, T. F., McGraw-Hill, Inc., 1989, p. 2649-2680). Though it is not precisely known how the mutation of the CFTR gene leads to the clinical manifestation (Welsh, M. J. et al. Cell 73:1251-1254, 1993), defective Cl⁻ secretion and increased Na⁺ absorption (Welsh, M. J. et al., Cell 73:1251-1254, 1993; Quinton, P. M., FASEB Lett. 4:2709-2717, 1990) are well documented. Furthermore, these changes in ion transport produce alterations in fluid transport across surface and gland epithelia (Jiang, C. et al., Science 262:424-427, 1993; Jiang, C. et al., J. Physiol. (London), 501.3:637-647, 1997; Smith, J. J. et al. J. Clin.l 1nvest., 91:1148-1153, 1993; and Zhang, Y. et al., Am.J.Physiol 270:C1326-1335, 1996). These resultant alterations in water and salt content of airway surface liquid (ASL) may diminish the activity of bactericidal peptides secreted from the epithelial cells (Smith, J. J. et al., Cell, 85:229-236, 1996) and/or impair mucociliary clearance, thereby promoting recurrent lung infection and inflammation.

Several lines of evidence suggest that submucosal glands contribute to the pathophysiology of CF lung disease. Maintenance of mucociliary clearance requires the coordinate regulation of ciliary motion, ASL depth, and mucin content. The quantity and composition of ASL are controlled by both the epithelium and submucosal glands and based on estimates of cell volume it appears that the latter may be a more important source of mucous secretions. Recent studies also indicate that the serous cells of the secretory tubules of the submucosal glands are the predominant site of CFTR expression in human bronchus and that fluid secreted from serous cells flushes out mucins secreted by mucous cells. Additional evidence suggesting that submucosal glands contribute to the pathophysiology of CF lung disease includes: (1) CFTR is predominantly expressed in the serous cells of the submucosal glands (Engelhardt, J. F. et al., Nat.Genet. 2:240-248, 1992), (2) tracheal submucosal gland cultures from CF patients fail to secret Cl⁻ (Finkbeiner, W. E., et al., Am.J.Physiol. 267:L-206-L-210, 1996; Yamaya, M., et al., Am.J.PhysioL 261:L-485-L-490, 1991; Yamaya, M., et al., Am.J.Physiol.261:L-491-L-494, 1991), (3) more than 60% of submucosal gland cultures from non-CF subjects showed a baseline secretion whilst cultures from CF patients exclusively absorbed fluid (Jiang, C., et al., J. Physiol. (London), 501.3:637-647, 1997), (4) obstruction of submucosal gland ducts is the first pulmonary manifestation in CF patients, and is followed by marked hyperplasia and hypertrophy (Oppenheimer, E. H. et al., New York: Year Book Medical Publishers, 1975, p. 241-278).

The evidence implicating submucosal glands in CF pathogenesis suggests that effective gene therapy for CF lung disease should target these structures. Though numerous attempts have been made to transfer the CFTR gene to surface epithelium, little attention has been paid to the submucosal gland cells. Additionally, while it has been demonstrated that low levels of β-galactosidase expression following intratracheal administration of adenovirus vectors were detectable in submucosal glands (Pilewski, J. M., et al., Am.J.Physiol. 268:L657-665, 1995), gland transfection levels were lower than for surface epithelium, and declined markedly with distance from the airway lumen.

Effective introduction of many types of biologically active molecules has been difficult and not all the methods that have been developed are able to effectuate efficient delivery of adequate amounts of the desired molecules into the targeted cells. The complex structure, behavior, and environment presented by an intact tissue that is targeted for intracellular delivery of biologically active molecules often interferes substantially with such delivery. Numerous methods, including viral vectors, DNA encapsulated in liposomes, lipid delivery vehicles, and naked DNA have been employed to deliver DNA into the cells of mammals. To date, delivery of DNA in vitro, ex vivo, and in vivo has been demonstrated using many of the aforementioned methods.

Though viral transfection is relatively efficient, the host immune response frequently posses a major problem. Specifically, viral proteins activate cytotoxic T lymphocytes (CTLs) which destroy the virus-infected cells thereby terminating gene expression in the lungs of in vivo models examined. The other problem is diminished gene transfer upon repeat administration of viral vectors due to the development of antiviral neutralizing antibodies. These issues are presently being addressed by modifying both the vectors and the host immune system. Additionally, non-viral and non-proteinaceous vectors have been gaining attention as alternative approaches.

Because compounds designed to facilitate intracellular delivery of biologically active molecules must interact with both non-polar and polar environments (in or on, for example, the plasma membrane, tissue fluids, compartments within the cell, and the biologically active molecule itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One group of amphiphilic compounds that have showed particular promise for efficient delivery of biologically active molecules are cationic amphiphiles. Cationic amphiphiles have polar groups that are capable of being positively charged at or around physiological pH, and this property is understood in the art to be important in defining how the amphiphiles interact with the many types of biologically active molecules including, for example, negatively charged polynucleotides such as DNA.

Examples of cationic amphiphilic compounds that are stated to be useful in the intracellular delivery of biologically active molecules are found, for example, in the following references, the disclosures of which are specifically incorporated by reference. Many of these references also contain useful discussions of the properties of cationic amphiphile that are understood in the art as making them suitable for such applications, and the nature of structures, as understood in the art, that are formed by complexing of such amphiphiles with therapeutic molecules intended for intracellular delivery.

(1) Felgner, et al., Proc. Natl. Acad. Sci. USA, 84, 7413-7417 (1987) disclose use of positively-charged synthetic cationic lipids including N-[1(2,3-dioleyloxy)propyl]-N,N,N-trimethylammonium chloride (“DOTMA”), to form lipid/DNA complexes suitable for transfections. See also Felgner et al., The Journal of Biological Chemistry, 269(4), 2550-2561 (1994).

(2) Behr et al., Proc. NatI. Acad. Sci., USA 86, 6982-6986 (1989) disclose numerous amphiphiles including dioctadecylamidologlycylspermine (“DOGS”).

(3) U.S. Pat. No. 5,283,185 to Epand et al. describe additional classes and species of amphiphiles including 3β[N-(N¹,N¹-dimethylaminoethane)-carbamoyl] cholesterol, termed “DC-chol”.

(4) Additional compounds that facilitate transport of biologically active molecules into cells are disclosed in U.S. Pat. No. 5,264,618 to Felgner et al. See also Felgner et al., The Journal of Biological Chemistry 269(4), pp. 2550-2561 (1994) for disclosure therein of further compounds including “DMRIE” 1,2-dimyristyloxypropyl-3-dimethyl-hydroxyethyl ammonium bromide, which is discussed below.

(5) Reference to amphiphiles suitable for intracellular delivery of biologically active molecules is also found in U.S. Pat. No. 5,334,761 to Gebeyehu et al., and in Feigner et al., Methods (Methods in Enzymology), 5, 67-75 (1993).

(6) Brigham, K. L., B. Meyrick, B. Christman, M. Magnuson, G. King and L. C. Berry. In vivo transfection of murine lungs with functioning prokaryotic gene using a liposome vehicle Am.J.Med. Sci. 298:278-281, 1989.

(7) Gao, X. A. and L. Huang. A novel cationic liposome reagent for efficient transfection of mammalian cells. Biochem Biophys Res Commun 179:280-285, 1991.

(8) Yoshimura, K., M. A. Rosenfeld, H. Nakamura, E. M. Scherer, A. Pavirani, J. P. Lecocq and R. G. Crystal. Expression of the human cystic fibrosis transmembrane conductance regulator gene in the mouse lung after in vivo intratracheal plasmid-mediated gene transfer. Nucl.Acids Res. 20:3233-3240, 1992.

(9) Zhu, N., D. Liggitt, Y. Liu and R. Debs. Systemic gene expression after intravenous DNA delivery into adult mice. Science 261:209-211, 1993.

(10) Solodin, I., C. S. Brown, M. S. Bruno, C. Y. Chow, E. Jang, R. J. Debs and T. D. Heath. A novel series of amphiphilic imidazolinium compounds for in vitro and in vivo gene delivery. Biochem. 34:13537-13544, 1995.

(11) Lee, E. R., J. Marshall, C. S. Siegal, C. Jiang, N. S. Yew, M. R. Nichols, J. B. Nietupski, R. J. Ziegler, M. Lane, K. X. Wang, N. C. Wan, R. K. Scheule, D. J. Harris, A. E. Smith and S. H. Cheng. Detailed analysis of structure and formulations of cationic lipids for efficient gene transfer to the lung. Hum.Gene Ther. 7:1701-1717, 1996.

Additionally, several recently issued U.S. Patents, the disclosures of which are specifically incorporated by reference herein, have described the utility of cationic amphiphiles to deliver polynucleotides to mammalian cells. (U.S. Pat. No. 5,676,954 to Brigham et al. and U.S. Pat. No. 5,703,055 to Feigner et al.)

Although the compounds mentioned in the above-identified references have been demonstrated to facilitate the entry of biologically active molecules into cells, it is believed that the uptake efficiencies provided thereby could be improved to support numerous therapeutic applications, particularly gene therapy. Additionally, it is sought to improve the activity of the above-identified compounds so that lesser quantities thereof are necessary, leading to reduced concerns about the toxicity of such compounds or of the metabolites thereof.

Another class of cationic amphiphiles with enhanced activity is described, for example, in U.S. Pat. No. 5,747,471 to Siegel et al. issued May 5, 1998, U.S. Pat. No. 5,650,096 to Harris et al. issued Jul. 22, 1997, and PCT publication WO 98/02191 published Jan. 22, 1998, the disclosures of which are specifically incorporated by reference herein. These patents also disclose formulations of cationic amphiphiles of relevance to the practice of the present invention.

While there are many cationic amphiphiles and viral vectors that have produced enhanced activity, new methods of binding and targeting lipid and non-lipid delivery vehicles to specific mammalian cells are still sought. A highly desired factor in using cationic amphiphiles and viral vectors for gene therapy, and other applications of in vivo, in vitro and ex vivo delivery of biologically active molecules, is the ability to effectively target and bind to specific mammalian cells. To date, effective methods which target specific cell types have been lacking. The ability to target specific cells would reduce the dosage of cationic amphiphile, viral or other delivery vehicle complexes needed to effectively treat a specific disease state thereby reducing the toxicity problems which are a function of higher doses. Consequently, methods of improving the efficiency and the quantity of biologically active molecules delivered to a desired mammalian cell are desired to enhance the viability of cationic amphiphile complexes, viral vectors, and other delivery vehicles as successful therapeutic treatments.

Accordingly, the present invention is directed to novel micellar complexes that facilitate delivery of biologically active molecules to the cells of a mammal. The novel micellar complexes are comprised of a cationic amphiphile, a biologically active molecule, a derivative of polyethylene glycol (PEG), and optionally, a neutral, positive, or negative co-lipid. These novel micellar complexes can possess unique properties that are not observed for traditional cationic amphiphile complexes. For example, the novel micellar complexes enable one skilled in the art to preferentially bind the micellar complex to airway epithelial cells. It may also be possible for the skilled artisan to preferentially bind the micellar complex to other specific cell types or to enable targeting of a specific mammalian cell for delivery by the micellar complex.

The present invention provides for the use of a cationic amphiphile to form a mixed micelle complex with a PEG derivative and optionally a co-lipid. All cationic amphiphiles that are capable of facilitating intracellular delivery of biologically active molecules are useful in the practice of the invention. Although the invention is not limited to the amphiphiles disclosed, numerous examples of cationic amphiphiles useful in the practice of the invention are described in the previously referenced publications.

In the practice of the invention, a micellar complex may be provided wherein the complex is effective for binding to airway epithelial cells. Not to be limited as to theory, it is believed that the micellar complexes demonstrate preferential binding as compared to traditional lipid complexes because of the difference in the charge density of the micellar complex.

The micellar complexes of the present invention may also be provided wherein the complex is substantially more homogeneous when compared to the traditional lipid complexes. In other words, micellar complexes of the present invention have a narrower size distribution curve than lipid complexes prepared by traditional means.

The preferred micellar complexes of the present invention are also more stable than traditional lipid complexes. A micellar formulation may be prepared the previous day and stored over night without any adverse affects.

In a further aspect, the invention provides for the improved efficiency of binding between the cationic amphiphile and the biologically active molecule. The improved efficiency of binding results in a higher amount or greater “loading” of DNA per lipid present in a formulation. It is known in the art that PEG derivatives stabilize a traditional lipid:biologically active molecule complex and prevent precipitation. However, the micellar complexes, which contain a PEG derivative, are able to load more biologically active molecule without precipitation than the traditional lipid bilayer complexes that also contain a PEG derivative. In other words, more biologically active molecules are associated with each cationic amphiphile in a micellar complex as compared to cationic amphiphiles in traditional cationic amphiphile complexes.

In a still further aspect, the invention includes a method of making a micellar lipid complex comprising a cationic amphiphile, a biologically active molecule, a PEG derivative, and optionally a co-lipid. The resulting complex is homogeneous, stable and effective for binding to airway epithelial cells. In a preferred embodiment, the complex is effective for systemic delivery of a biologically active molecule.

The invention also provides for a method of delivering a biologically active molecule to a mammalian cell by administering a micellar complex. Additionally, a method is provided to facilitate transfection of a gene to a mammalian cell by administration of a micellar complex.

In a still further aspect of the invention, the micellar complexes may also include a targeting agent that facilitates delivery of a biologically active molecule to a specific type of mammalian cell. The targeting agents are effective for both lipid and non-lipid methods and the invention provides for use of targeting agents in all lipid complexes, including both traditional and micellar cationic amphiphiles, along with the use of targeting agents in viral vectors including adenoviruses, and other methods that have been employed in the art to effectuate delivery of biologically active molecules into the cells of mammals.

The invention also provides for pharmaceutical compositions of micellar complexes and pharmaceutical compositions of other lipid and non-lipid complexes with targeting agents. The micellar complexes may be the active ingredient in a pharmaceutical composition that includes carriers, fillers, extenders, dispersants, creams, gels, solutions and other excipients that are common in the pharmaceutical formulatory arts.

Additional features and advantages of the invention will be set forth in the description which follows, and in part will be apparent from the description, or may be learned by practice of the invention. The objectives and other advantages of the invention will be realized and attained by the method particularly pointed out in the written description and claims herein as well as the appended drawings.

### BRIEF DESCRIPTION OF THE DRAWINGS

FIG. 1. depicts a procedure for the formulation of traditional lipid complexes (a) compared to micellar complexes with (b) and without (c) a co-lipid.

FIG. 2. depicts the size distribution of a traditional cationic lipid GL-67:pDNA complex (a) (GL-67:pDNA (0.5:0.5) & GL-67:DOPE:DMPE-PEG5000 (1:2:0.05)) compared to the size distribution of micellar complexes ((b) & (c)). (b) (GL-67:DMPE-PEG5000:pDNA (1.5:0.5:2)) represents the size distribution of a micellar complex lacking the minimum amount of PEG necessary to form the preferred homogeneous complex, while (c) (GL-67:DMPE-PEG5000:pDNA (1.5:0.75:2)) depicts the size distribution of a micellar complex prepared with a sufficient amount of PEG.

FIG. 3. depicts the size distribution of a traditional cationic lipid GL-89:pDNA complex (a) (GL-89:pDNA (2:2) & GL-67:DOPE:DMPE-PEG5000 (1:1:0.005)) compared to the size distribution of micellar complexes ((b) & (c)). (b) (GL-89:DMPE-PEG5000:pDNA (1.5:0.0025:2)) represents the size distribution of a micellar complex lacking the minimum amount of PEG necessary to form the preferred homogeneous complex, while (c) (GL-67:DMPE-PEG5000:pDNA (1.5:0.25:2)) depicts the size distribution of a micellar complex prepared with a sufficient amount of PEG.

FIG. 4. depicts the change in size distribution of a micellar cationic lipid GL-67:pDNA complex as the amounts of co-lipid and PEG (DOPE:DMPE-PEG₅₀₀₀) are increased. A minimum amount of PEG is necessary to form the small homogeneous and stable micellar complexes.

In the present invention, cationic amphiphile compounds of the prior art are used in formulations containing a PEG derivative and optionally a co-lipid. The resulting formulations are complexed to one or more biologically active molecules. The novel formulations exhibit unique and surprising properties that are not found in traditional cationic amphiphile formulations, other cationic amphiphile formulations, and lipid carriers. An additional aspect of the invention is the use of targeting agents in the new formulations. The targeting agents facilitate delivery to specific mammalian cells. The practice of the invention is not limited as to theory.

Traditional complexes of a cationic amphiphile, a PEG derivative, and optionally a co-lipid are well known in the art. These traditional complexes are formed by preparing a lipid film of the cationic amphiphile, the PEG derivative, and optionally the co-lipid. The lipid film is then hydrated in aqueous media to form a lipid bilayer which is then complexed to a biologically active molecule. Traditional cationic amphiphile complexes formed via this method are normally 400-500 nm (nanometers) in diameter and vary in size by 50% or greater.

A preferred embodiment of the present invention is a small, homogenous, and stable mixed micelle formulation or micellar complex. One embodiment of the invention contemplates a micellar complex that exhibits binding to airway epithelia cells, a property not found with traditional cationic amphiphile complexes. In the practice of the present invention a micellar complex formulation that can have unique and surprising properties is prepared via a new method. The micellar complex may preferably be prepared by hydrating the cationic lipid and adding the hydrated cationic lipid to the PEG derivative which has also been hydrated in order to form a micellar lipid suspension. The micellar cationic lipid:PEG:biologically active molecule complex is prepared by adding the micellar cationic lipid:PEG derivative solution to the biologically active molecule. The molar ratio of lipid:biologically active molecule and of cationic lipid:PEG derivative may vary over a wide range and will depend on the cationic lipid, PEG derivative, and biologically active molecule that is being utilized. The ratios may also vary significantly as a function of administration site and disease target. In an embodiment, the molar ratio of lipid:biologically active molecule is 1:8. In a further preferred embodiment the biologically active molecule is DNA.

A micellar complex may also be prepared with a neutral, positive, or negative co-lipid as part of the formulation. The co-lipid is formulated with the PEG lipid as a lipid film and hydrated as a single solution or the co-lipid can be formulated alone as a lipid film and hydrated with PEG lipid. The cationic lipid is then added in hydrate form to the PEG lipid and co-lipid solution to form a micellar lipid which may then be used to form a micellar complex with a biologically active molecule. In an embodiment, the molar ratio of lipid:biologically active molecule is 1:8. In a further preferred embodiment the biologically active molecule is DNA.

In the practice of the invention, a micellar lipid complex may be provided wherein the complex is effective for binding to airway epithelial cells. Not to be limited as to theory, it is believed that the micellar lipid complexes demonstrate preferential binding as compared to traditional lipid complexes because of the difference in the charge density of the micellar complex.

The micellar complexes of the present invention may also be provided wherein the complex is substantially more homogeneous when compared to the traditional lipid complexes. In other words, in this embodiment, micellar complexes of the present invention have a narrower size distribution curve than lipid complexes prepared by traditional means. For example, the size distribution of a traditional lipid complex may vary by greater than 50% depending on the lipid, the DNA, and the lipid:DNA ratio. By comparison, the size distribution of micellar complexes in accord with this embodiment may only vary by a maximum of about 20%.

In addition to being significantly more homogeneous, the preferred micellar complexes of the present invention may not appreciably vary in size upon the addition of more biologically active molecules to a complex. For example, experiments were preformed in which the ratios in micellar complexes of cationic lipid to pDNA and of cationic lipid to PEG derivative to co-lipid were constant while the amount of pDNA that was a part of the micellar complexes was increased (i.e., the pDNA was not free in solution). The size of the preferred micellar complexes and their size distribution did not vary significantly as the amount of pDNA in the micellar complexes was increased.

The preferred micellar complexes of the present invention are also more stable than traditional lipid complexes. Many traditional lipid complexes experience storage and stability problems which require special storage procedures or mixing of the formulation with the DNA to be delivered immediately before administration to a mammal or to cells in vitro. For example traditional cationic lipids are known to degrade via transacylation reactions unless stored under specific conditions. Many traditional lipid:DNA complexes are also known to precipitate out of solution shortly after complex formation therefore requiring a postponement of the preparation of the complexes until immediately before use. A pharmaceutical product which requires the formulation to be made immediately before use is not very practical. Micellar complexes of the present invention preferably do not precipitate out of solution shortly after formulation. For example, a micellar formulation may be prepared the previous day and stored over night without any adverse affects. One of ordinary skill in the art may also vortex a micellar formulation without observing significant precipitation.

A minimum amount of PEG lipid is preferred to form a stable, homogeneous complex when the micellar lipid solution is added to the biologically active molecule. The minimum amount of PEG needed is dependent upon the specific combination of cationic lipid and PEG lipid selected. Methods to determine the minimum amount of PEG required to form the micellar complex may include but are not limited to: 1) Observation of the lipid:biologically active molecule complex following addition of the micellar lipid to the biologically active molecule to verify that the suspension is clear to opaque and lacks particulates; 2) Sizing of the micellar lipid:biologically active molecule complex following preparation using a particle sizer in order to determine whether the particle population is substantially homogeneous with regard to particle size; and 3) Analysis of the behavior of the biologically active molecule in the micellar complex in agarose gel electrophoresis. More detail regarding the above mentioned methods can be found in the examples enclosed herewith.

Another embodiment of the invention relates to micellar complexes that are smaller in diameter than traditional cationic amphiphile complexes and remain small and stable throughout a wide range of lipid:DNA and lipid:PEG ratios. A minimum amount of PEG derivative is preferred to form small, homogeneous micellar complexes. In a preferred embodiment of the invention, a micellar complex prepared with one or more cationic amphiphiles, one or more PEG derivatives, a biologically active molecule, and optionally a co-lipid, is on average approximately 25 to 250 nanometers in diameter. However the size of a micellar complex is dependent on the cationic lipid or lipids employed, the PEG lipid or lipids, the amount and size of the DNA, and the co-lipid, if present.

Not to be limited as to theory, the charge density of the micellar complexes is believed to be responsible for the preferred unique and surprising property of the complexes to bind to airway epithelial cells. It is believed that the higher charge density translates into a higher affinity for certain cell membranes. Consequently, many of the micellar complexes bind to airway epithelial cells. A simple in vitro fluorescence experiment demonstrates that micellar complexes appreciably bind to exposed airway epithelial cells. Traditional complexes of cationic amphiphiles, normally 400-500 nm in diameter, do not exhibit appreciable binding in the same experiment.

The micellar complexes of the present invention are also believed to be less toxic upon administration to a mammal than traditional cationic lipid complexes. For example, when injected intravenously into mice, micellar complexes prepared using cationic lipid GL-67 were less toxic than traditional cationic lipid complexes also prepared using GL-67. The lower toxicity of the micellar complexes does not significantly affect the complexes ability to deliver to tumor cells. In a preferred embodiment, the micellar complexes maintain a comparable deposition in tumor cells and specifically tumor endothelial cells while they are less toxic in regard to other cells of a mammal.

In another embodiment of the present invention, the micellar complexes are coated by lipids or other compositions used in the pharmaceutical arts to coat compositions and formulations. For example, mixing a micellar complex with a further hydrophobic species, such as a neutral lipid mixture, may coat the outside of the complex without disturbing the complex. Not being limited as to theory, it is desirably to use the cationic lipid:PEG derivative to condense DNA efficiently. The resulting small, highly condensed package of DNA, e.g., the micellar complex, can then be surrounded by other species and lipids, such as co-lipids or other PEG derivatives, which could interact with the charged surface of the condensed DNA. This may protect and/or mask the cationic lipid:DNA from the immune system. Since much of the toxicity of lipid:DNA complexes is due to bacterial sequence recognition, coating may be a valuable tool to reducing toxicity. Additionally, the coating may facilitate the inclusion of a targeting agent allowing delivery of the complex to a specific tissue or cell type. In a preferred embodiment, the coated complex is delivered systemically.

Further, coatings may be useful in order to extend the residence time of a micellar complex in the blood stream or as a time-release mechanism. Other coatings or uses of coatings known in pharmaceutical arts are within the practice of the invention.

Cationic Amphiphiles for use in Micellar Complexes

This invention provides for the use of any cationic amphiphile or cationic lipid compounds, and compositions containing them, that are useful to facilitate delivery of biologically active molecules to cells. Amphiphiles that are particularly useful facilitate the transport of biologically active polynucleotides into cells, and in particular to the cells of patients for the purpose of gene therapy.

A number of preferred cationic amphiphiles according to the practice of the invention can be found in U.S. Pat. Nos. 5,747,471 & 5,650,096 and PCT publication WO 98/02191, the disclosures of which are specifically incorporated by reference herein. In addition to cationic amphiphile compounds, these two patents disclose numerous preferred co-lipids, biologically active molecules, formulations, procedures, routes of administration, and dosages.

In connection with the practice of the present invention, cationic amphiphiles tend to have one or more positive charges in a solution that is at or near physiological pH. Representative cationic amphiphiles that are useful in the practice of the invention are:

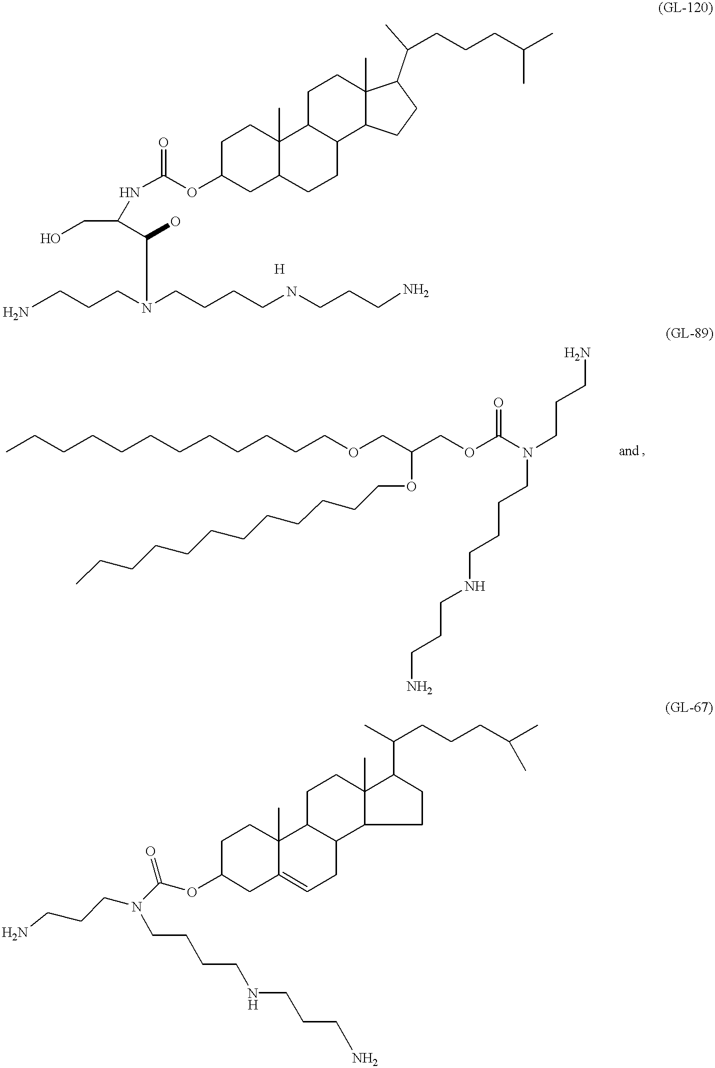

and other amphiphiles as are known in the art including those described in U.S. Pat. No. 5,747,471, the disclosure of which is specifically incorporated by reference herein.

PEG Derivatives

As discussed above, it has been surprisingly determined that the stability of cationic amphiphile compositions (both traditional and micellar) can be substantially improved by adding to such formulations small additional amounts of one or more derivatized polyethylene glycol compounds. Such enhanced performance is particularly apparent when measured by stability of cationic amphiphile formulations to storage and manipulation.

PEG derivatives were originally used to stabilize traditional cationic amphiphile formulations. Not to be limited as to theory, the use of PEG and PEG derivatives enables one to use a higher ratio of lipid to DNA. Previous attempts to prepare more concentrated lipid:pDNA complexes using traditional formulations resulted in precipitation of the complexes, especially at lipid:pDNA ratios for which the majority of the PDNA was bound to lipid. It was believed that the precipitation observed at higher concentrations in traditional formulations might be related to a phase separation of the cationic lipid component from the non-bilayer lipid component. In an attempt to maintain the traditional lipid formulations in a bilayer configuration, PEG-containing lipids were found to be effective in preventing precipitation of the complex at higher pDNA concentrations.

Only a small mole fraction of PEG-containing lipid was used to form stable traditional formulations that did not precipitate at high concentrations of lipid and DNA. For example, at 1.6 mol % PEG-DMPE, cationic lipid:pDNA complexes could be stabilized at pDNA concentrations exceeding 20 mM. For more information regarding use of PEG derivatives the following references are specifically incorporated by reference. Simon J. Eastman et al., Human Gene Therapy, 8, pp. 765-773 (1997); Simon J. Eastman et al. Human Gene Therapy, p. 8, pp. 313-322 (1997).

It was subsequently determined that a PEG derivative could be also used to prepare novel micellar formulations. The PEG containing formulations of the micellar complexes can exhibit unique properties not found with traditional formulations of cationic amphiphiles that also contain PEG derivatives including improvement in the affinity of the formulations to biologically active molecules.

The improved efficiency of binding results in a higher amount or greater “loading” of DNA per lipid present in a formulation. It is known in the art that PEG derivatives stabilize the lipid:biologically active molecule complex and prevent precipitation. However, the micellar complexes, which contain a PEG derivative, are able to load more biologically active molecule without precipitation than the traditional lipid bilayer complexes that also contain a PEG derivative. In other words, more biologically active molecules are associated with each cationic amphiphile in a micellar complex as compared to cationic amphiphiles in traditional cationic amphiphile complexes. For example, at a 0.125:1 molar ratio of amphiphile:pDNA all of the pDNA appears to be associated with the micellar complexes. Traditional cationic amphiphile complexes require a 0.75:1 to 1:1 molar ratio of amphiphile:pDNA to completely bind all of the pDNA. The high affinity for pDNA of the micellar systems enables one to deliver much more pDNA using fewer cationic amphiphiles.

In regard to micellar complexes, a minimum amount of PEG lipid can form a stable, homogeneous complex when the micellar lipid solution is added to the biologically active molecule. According to the practice of the invention, any derivative of polyethylene glycol may be part of the formulation to prepare a micellar complex. Complexes have been prepared using a variety of PEG derivatives and all of the PEG derivatives, at a certain minimum cationic amphiphile:PEG derivative ratio have been able to form small, homogeneous complexes. The micellar complexes remain stable and homogeneous through a wide range of cationic lipid:PEG and cationic lipid:DNA ratios once the minimum amount of PEG lipid to form the small, homogeneous complexes is determined. The minimum amount of PEG to form the stable, homogeneous complex may be routinely determined by the skilled artisan.

The minimum amount of PEG used is dependent upon the specific combination of cationic lipid and PEG lipid selected. For example, cationic lipids with an acyl chain (GL-89) are less likely to precipitate upon mixing with biologically active molecules than cholesterol-based lipids such as GL-67. This is not to suggest that cholesterol-based lipids such as GL-67 are ineffective, but only that a different ratio of cationic lipid:PEG derivative is used to form stable, homogeneous, micellar complexes. Consequently, one might choose a cationic lipid that is stable enough to form a micellar formulation without the presence of a PEG derivative.

Derivatives of polyethylene glycol useful in the practice of the invention include any PEG polymer derivative with a hydrophobic group attached to the PEG polymer. Examples would include PEG-DSPE, PEG-PE, PEG-DMPE, PEG-DOPE, PEG-DPPE, or PEG-ceramide. Not to be limited as to theory, it is believed that preferred PEG-containing lipids would be any PEG polymer derivatives attached to a hydrophobic group that can stabilize/interact with a cationic lipid. Two highly preferred species thereof include dimyristoylphosphatidylethanolamine (di C₁₄) (“DMPE”); and distearoylphosphatidylethanolamine (di C₁₈) (“DSPE”).

With respect to selection of the PEG polymer, it is a preferred embodiment of the invention that the polymer be linear, having a molecular weight ranging from 1,000 to 10,000. Preferred species thereof include those having molecular weights from 1500 to 7000, with 2000 and 5000 being examples of useful, and commercially available sizes. In the practice of the invention, it is convenient to use derivatized PEG species provided from commercial sources, and it is noted that the molecular weight assigned to PEG in such products often represents a molecular weight average, there being shorter and longer molecules in the product. Such molecular weight ranges are typically a consequence of the synthetic procedures used, and the use of any such product is within the practice of the invention.

It is also within the practice of the invention to use derivatized-PEG species that (1) include more than one attached phospholipid, or (2) include branched PEG sequence, or (3) include both of modifications (1) and (2).

Accordingly, preferred species of derivatized PEG include:

(a) polyethylene glycol 5000-dimyristoylphosphatidylethanolamine, also referred to as PEG(₅₀₀₀)-DMPE;

(b) polyethylene glycol 2000-dimyristoylphosphatidylethanolamine, also referred to as PEG(₂₀₀₀)-DMPE);

(c) polyethylene glycol 5000-distearoylphosphatidylethanolamine, also referred to as PEG(₅₀₀₀)-DSPE); and

(d) polyethylene glycol 2000-distearoylphosphatidylethanolamine, also referred to as PEG(₂₀₀₀-DSPE).

Certain phospholipid derivatives of PEG may be obtained from commercial suppliers. For example, the following species: di C14:0, di C16:0, di C18:0, di C18:1, and 16:0/18:1 are available as average 2000 or average 5000 MW PEG derivatives from Avanti Polar Lipids, Alabaster, Ala., USA, as catalog nos. 880150, 880160, 880120, 880130, 880140, 880210, 880200, 880220, 880230, and 880240.

Selection of Co-lipids

The use of co-lipids is optional. Depending on the formulation, including neutral, positive, or negative co-lipids in the micellar complex may substantially enhance delivery and transfection capabilities. Representative co-lipids include dioleoylphosphatidylethanolamine (“DOPE”), the species most commonly used in the art, diphytanoylphosphatidylethanolamine, lyso-phosphatidylethanolamines other phosphatidyl-ethanolamines, phosphatidylcholines, lyso-phosphatidylcholines, phosphatidyl-inositol and cholesterol. Typically, a preferred molar ratio of cationic amphiphile to co-lipid is about 1:1.However, it is within the practice of the invention to vary this ratio, including also over a considerable range, although a ratio from 2:1 through 1:2 is usually preferable. Use of diphytanoylphosphatidylethanolamine is highly preferred according to the practice of the present invention, as is use of “DOPE”.

According to the practice of the invention, preferred formulations may also be defined in relation to the mole ratio of PEG derivative, however, the preferred ratio will vary with the cationic amphiphile chosen. A representative preferred micellar formulation according to the practice of the present invention has the cationic amphiphile GL-67: co-lipid DOPE: PEG₅₀₀₀ molar composition ratio of about 1:1:0.25. In preferred examples thereof, the co-lipid is diphytanoylphosphatidylethanolamine, or is DOPE, and the PEG derivative is a DMPE or DSPE conjugate of PEG₂₀₀₀ or PEG₅₀₀₀.

Biologically Active Molecules

Biologically active molecules that can be useful in the practice of the invention include, for example, genomic DNA, cDNA, mRNA, antisense RNA or DNA, oligodeoxynucleotides, polypeptides and small molecular weight drugs or hormones. In the practice of the invention, one skilled in the art can as a matter of routine experimentation determine which molecules will be effectively delivered to a mammalian cell. It is well known in the art that once delivery of a biologically active molecule by a cationic amphiphile complex (or other lipid or non-lipid carriers) to a mammalian cell is demonstrated, the choice of other molecules for delivery is routine.

Targeting and Targeting Complexes

A further aspect of the invention is the use of targeting agents in any of the methods that effectuate the delivery of biologically active molecules into the cells of mammals. In a preferred embodiment, targeting agents are used with both traditional and micellar cationic amphiphile formulations or viral formulations such as viral vectors and adenoviruses. A targeting agent is usually a molecule, peptide sequence, or large protein that preferentially targets or binds to specific mammalian cells. Many targeting agents are molecules that are well known in the art. For example, Pertactin, a peptide containing the RGD sequence, preferentially targets and binds to airway epithelial cells. In the practice of the invention, a targeting agent or ligand is attached to a carrier complex. It is well known in the art that although a lone targeting agent will target specific cells, attachment of a targeting agent to another entity will often alter or destroy the molecule's targeting activity. However, attachment of a targeting agent to a PEG derivative does not always destroy targeting agent activity. Therefore, attachment of a targeting agent to a micellar complex may also preserve the agent's activity, and it is a preferred embodiment of the invention to attach a targeting agent to a micellar complex.

Coupling of a targeting agent to a cationic amphiphile complex, adenovirus, or other carrier will enable specific targeting to desired mammalian cells. Advantageously, targeting to a desired mammalian cell will enable more efficient delivery of biologically active molecules and therefore increase transfection of the targeted mammalian cell.

A lipid complex coupled to a targeting agent may comprise any cationic amphiphile as described above, a PEG derivative, a biologically active molecule, and optionally a co-lipid. Additionally, there may be formulations in which the PEG derivative is not necessary and the targeting agent is coupled directly to a cationic amphiphile/biologically active molecule complex with optionally a co-lipid. The lipid complex may be a micellar or traditional lipid formulation. The targeting agent may also be appended directly to the PEG derivative, i.e., PEG-DMPE. In a further embodiment, the targeting agent is coupled to a cationic polymer or to a hydrophobic moiety such as a lipid.

Any targeting agent known in the art may be useful in the practice of the invention. Preferred targeting agents include: Pertactin, a peptide containing an RGD sequence that targets airway epithelial cells; UDP/UTP, which targets the P2U receptors including P2U receptors on cells in the airways; Lactose, which targets endogenous lectins in airways and the liver; and Cyclic RGD peptide, which targets tumor endothelial cells. Other preferred targeting agents include Penetratin, an amphiphilic peptide, lectins, agents to target the LDL receptor, mannose-6-phosphate which targets the mannose-6-PO₄ receptor, and airway specific single chain antibodies.

In a preferred aspect of the invention, peptide ligands can be incorporated into the lipid:biologically active molecule complexes to augment the transfection activity of the gene transfer system or to improve binding to airway epithelial cells.

Other examples of peptide targeting agents include HAV peptides and CNP-22 peptides. HAV peptides are a series of peptides containing the sequence of histidine, alanine and valine that modulate cadherin-mediated cell adhesion. Not to be limited as to theory, cadherin complexes form cell-cell adhesion to maintain tissue integrity and generate physical and permeability barriers in the body. Cadherins have been shown to regulate epithelial, endothelial, neural and tumor cell adhesion. The cell adhesion is achieved through interactions between the extracelluar domains of cadherins between cells, and cytoplasmic domains of cadherin with the catenin proteins and the actin cytoskeleton within the cell. A tri-peptide of histidine, alanine and valine (HAV) is located in the extracellular and cytoplasmic domains of cadherin(s). The HAV peptide is crucial for hemophilic interactions between cadherins, and plays an important role in the interaction with actin cytoskeleton via the catenin proteins. HAV peptides may be linear or cyclic.

In the practice of the present invention, HAV peptides preferably bind to and becomes internalized by epithelial cells in airways and therefore may be utilized as targeting agents for delivering biologically active molecules to: 1) epithelial cells, for example, as a targeting agent to deliver the cystic fibrosis transmembrane conductance regulator (CFTR) gene to airway epithelial cells in CF lung; 2) endothelial cells, for example, inhibiting angiogenesis around a tumor by delivering a gene that can cause apoptosis for endothelial cells; 3) neural cells; and 4) tumor cells. HAV may also be chemically conjugated with, for example, 1) poly-L-lysine or linear/branched polyethylenimine or other polypeptides or (X)-phosphatidylethanolamine (X-PE; e.g., N-MPB-PE) through linker(s) and pDNA complexes with or without lipids; or 2) viral vectors through linkers, to deliver biologically active molecules more efficiently to targeted cells. One may also use conjugate, positively charged HAV for pre-treatment followed by administration of negatively charged non-viral or viral vectors, or co-administer the mixed complexes of conjugated, positively charged HAV with negatively charged non-viral or viral vectors.

HAV peptides may also be used as cell adhesion regulators. Some HAV peptides are more potent at disrupting cell-cell adhesion junctions, and some are more potent at preventing the formation of cell-cell adhesion junctions. Based on these functions, one may use the peptides alone, or combined with tight junction disrupting agents, like EGTA or palmitoyl-L-carnitine or dimethyl β-cyclodextrin or methyl β-cyclodextrin or α-cyclodextrin to improve delivery of a biologically active molecule to: 1) epithelial cells, for example, delivery of the CFTR gene through cell-cell permeability barriers in airway epithelial of CF lung to enhance gene uptake on the cell basolateral membrane; 2) endothelial cells, for example, delivery of genes through brain-blood barriers to tumor cells; 3) neural cells, for example, to increase vector migration; 4) tumor cells, especially solid tumors (e.g., melanomas) since many solid tumors develop internal barriers that limit the gene delivery to inner cells or cells distant from the injection site; and 5) muscle, liver or other whole organs by local injection with vector in order to increase vector migration.

C-type natriuretic peptide containing 22 amino acid (CNP-22) binds to and activates the guanylate cyclase-B (GC-B) receptor, a transmembrane receptor that contains intracellular guanylate cyclase domain. Not to be limited as to theory, the regulator pathway of the peptide is thought to be mediated predominantly through cyclic GMP (cGMP). In the lung, CNP-22 binding and function may predominate in airway epithelial cells. Specific binding of CNP-22 to airway epithelial cells in vivo has been demonstrated by the functional ability of CNP-22 to elevate cGMP levels, active CFTR-dependent chloride transport, and stimulate ciliary beat frequency. Additionally, CNP-22 conjugated with 16 lysine (K16-CNP-22) binds to some type(s) of epithelial cells in mouse trachea, and other airways.

In the practice of the invention, CNP-22 may be used as a targeting agent. For exmple, adenovirus vector (AdV) mediated gene transfer to mouse trachea, other airways, and lung are increased in mice treated with K16-CNP-22. Enhancement of cationic lipid:pDNA mediated gene transfer to the lung is also observed in mice treated with K16-CNP-22. Since CNP-22 and/or GC-B receptor have also been identified in brain, uterus/oviduct, small intestine, colon and kidney, CNP-22 peptides may also be used to target these organs for delivery of biologically active molecules.

It is also within the practice of the invention to chemically conjugate CNP with 1) poly-L-lysine, linear/branch polyethylenimine or other polypeptides or (X)-phosphatidylethanolamines (X-PE; e.g.: N-MPB-PE) through linker(s), and make pDNA complexes with or without lipids, or 2) viral vectors through linker(s), to deliver genes more efficiently to targeted cells. Conjugated, positively charged CNP may also be used for pre-treatment followed by administration of negatively charged non-viral or viral vectors, or co-administration of the mixed complexes of conjugated, positively charged CNP with negatively charged non-viral or viral vectors.

In the practice of the invention, traditional and micellar complexes containing targeting agents may be formulated and administered in tile same manner and using the same methods as complexes without targeting agents. Similarly, the ratio of cationic amphiphile:PEG derivative and cationic amphiphile:biologically active molecule would be dependent on the type of lipid used.

Preparation of Pharmaceutical Compositions and Methods of Administration

The present invention provides for pharmaceutical compositions that facilitate delivery and/or transfection of biologically active molecules. Pharmaceutical compositions of the invention facilitate delivery of biologically active molecules into tissues and organs such as the gastric mucosa, heart, lung, liver, and tumor vasculature, and solid tumors. Additionally, compositions of the invention facilitate entry of biologically active molecules into cells that are maintained in vitro, such as in tissue culture.

Biologically active molecules that can be provided intracellularly in therapeutic amounts using the amphiphiles of the invention include: (a) polynucleotides such as genomic DNA, cDNA, and mRNA that encode for therapeutically useful proteins as are known in the art; (b) ribosomal RNA; (c) antisense polynucleotides, whether RNA or DNA, that are useful to inactivate transcription products of genes and which are useful, for example, as therapies to regulate the growth of malignant cells; (d) ribozymes; and (e) low molecular weight biologically active molecules such as hormones and antibiotics.

Cationic amphiphile species, PEG derivatives, and co-lipids of the invention may be blended so that two or more species of cationic amphiphile or PEG derivative or co-lipid are used, in combination, to facilitate entry of biologically active molecules into target cells and/or into subcellular compartments thereof. Cationic amphiphiles of the invention can also be blended for such use with amphiphiles that are known in the art. Additionally, a targeting agent may be coupled to any combination of cationic amphiphile, PEG derivative, and co-lipid.

Dosages of the pharmaceutical compositions of the invention will vary, depending on factors such as half-life of the biologically-active molecule, potency of the biologically-active molecule, half-life of the amphiphile(s), any potential adverse effects of the amphiphile(s) or of degradation products thereof, the route of administration, the condition of the patient, and the like. Such factors are capable of determination by those skilled in the art.

A variety of methods of administration may be used to provide highly accurate dosages of the micellar complexes and pharmaceutical compositions containing micellar complexes of the invention. Such preparations can be administered orally, intravenously, parenterally, topically, transmucosally, or by injection of a preparation into a body cavity of the patient, or by using a sustained-release formulation containing a biodegradable material, or by onsite delivery using additional micelles, gels and liposomes. Nebulizing devices, powder inhalers, and aerosolized solutions are representative of methods that may be used to administer such preparations to the respiratory tract. It is also within the practice of the invention to use micellar complexes for systemic delivery.

Additionally, the therapeutic compositions of the invention can in general be formulated with excipients (such as the carbohydrates lactose, trehalose, sucrose, mannitol, maltose or galactose, and inorganic or organic salts) and may also be lyophilized (and then rehydrated) in the presence of such excipients prior to use. Conditions of optimized formulation for each complex of the invention are capable of determination by those skilled in the pharmaceutical art. Selection of optimum concentrations of particular excipients for particular formulations is subject to experimentation, but can be determined by those skilled in the art for each such formulation.

An additional aspect of the invention concerns the protonation state of the cationic amphiphiles of the complexes of the invention prior to their contacting the biologically active molecule for delivery, or prior to the time when said complex contacts a biological fluid. It is within the practice of the invention to provide fully protonated, partially protonated, or free base forms of the amphiphiles in order to form, or maintain, such therapeutic compositions.

### EXAMPLES

### Example 1

### Preparation of Micellar and Traditional Cationic Lipid:Biologicially Active Molecule Complexes

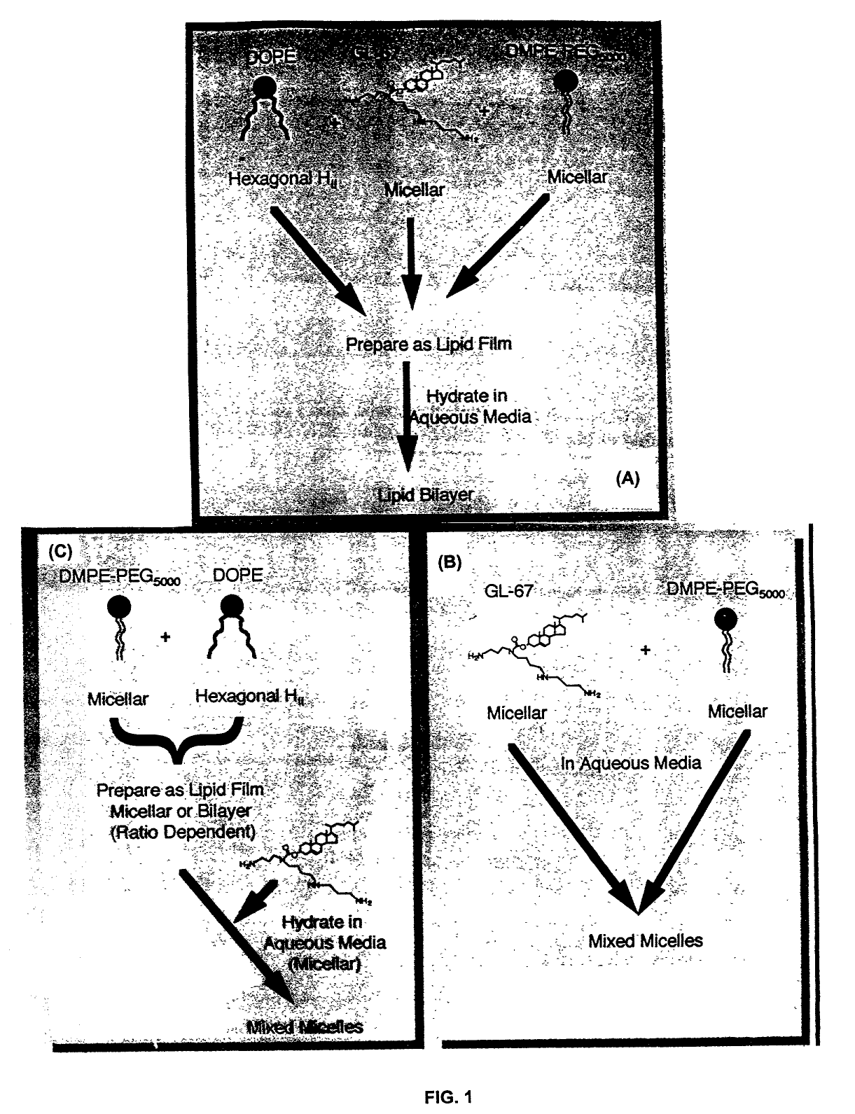

The following example outlines typical procedures used to prepare a cationic lipid micellar complex. FIG. 1 is a schematic representation that depicts a procedure for the formulation of traditional cationic lipid complexes (a) as compared to cationic lipid micellar complexes with (b) and without (c) a co-lipid. The practice of the present invention is not limited to the procedures disclosed herewith.

Preparation of cationic lipid:PEG lipid:PDNA micellar complex

(1) The micellar cationic lipid:PEG lipid solution was prepared as follows. The cationic lipid was hydrated at four times the concentration of the desired final cationic lipid concentration of the lipid:pDNA complex (a typical but not exclusive range is 0.25-16 mM cationic lipid). The PEG containing lipid was hydrated at four times the concentration of the desired final PEG lipid concentration of the lipid:pDNA complex (a typical but not exclusive range is 0.25-16 mM cationic lipid). (In regard to the PEG lipid, a full range of lipid anchors has been utilized and the PEG head group may be any one of a variety of sizes.) Once hydrated, the cationic lipid was added to the PEG lipid at a 1:1(vol:vol) ratio. While this is a typical method, it is not required as long as the desired ratio of cationic lipid:PEG lipid is ultimately achieved. The plasmid DNA was diluted to two times the desired final pDNA concentration of the lipid:pDNA complex. The cationic lipid:PEG lipid:pDNA complex was then prepared by adding the micellar cationic:PEG lipid solution to the pDNA at a 1:1 ratio (vol:vol).

(2) The micellar cationic lipid solution was also prepared with a co-lipid as part of the formulation. This was done as indicated above in (1) except that the co-lipid was formulated with the PEG lipid as a lipid film and hydrated as a single solution or in an alternative procedure the co-lipid was formulated as a lipid film and hydrated with PEG lipid. The PEG:co- lipid solution can then be substituted for the PEG lipid above.

Analysis of the Micellar Complex

A minimum amount of PEG lipid was preferably used to form a stable, homogeneous complex when the micellar lipid solution was added to the biologically active molecule. Additionally, the minimum amount of PEG needed was dependent upon the specific combination of cationic lipid and PEG lipid selected. Methods to determine the minimum amount of PEG used to form the micellar complex may include but are not limited to:

1) The lipid:biologically active molecule complex was observed following addition of the micellar lipid to the biologically active molecule. When the micellar complex was observed after preparation, the suspension was clear to opaque and lacked particulates. If particulates were observed, the formulation was lacking a minimum amount of PEG to form the preferred stable, homogeneous micellar complexes. By comparison, traditional cationic lipid:pDNA complexes were generally opaque solutions that did not have particulates in them.

2) The micellar lipid:biologically active complex was sized following preparation using a particle sizer. When the micellar complex was sized following preparation, the particle population was substantially homogeneous with regard to particle size and more preferably was small (in a preferred embodiment approximately 25-250 nm in diameter). If the size population contained large, heterogeneous particles, the minimum amount of PEG lipid was not present in the formulation. By comparison, traditional cationic lipid:pDNA complexes generally yielded particles that were around 200-800 nm in diameter. These suspensions tend to be quite heterogeneous in size and the size of the complex depended heavily on the cationic lipid used in the formulation. No traditional cationic lipid complexes were generally observed which exhibited the small, homogeneous characteristics observed with the micellar formulations.

3) The behavior of the biologically active molecule in the micellar complex was analyzed in agarose gel electrophoresis. If a minimum amount of PEG lipid to form a stable, homogeneous micellar complex was used, the biologically active molecule migrated into the agarose gel in a manner different from that of the free biologically active molecule (although it was possible to visualize a population of “free” plasmid in addition to the complexed plasmid). If the minimum amount of PEG lipid had not been used, the majority of the plasmid visualized in the gel either: 1) migrated like free pDNA or 2) was retained in the well of the gel and therefore was not visible in the gel. More than one of these tests was done in order to lend confidence that the minimum amount of PEG lipid had been used.

Analysis of the micellar lipid:pDNA complex by agarose gel electrophoresis was performed by preparing a 0.7% agarose gel in Tris-borate EDTA buffer pH 8.0. A volume of micellar lipid:pDNA complex which contained from 0.25-1 μg of pDNA was loaded per well. The gel was run for approximately 1 hour at 100 V. The gel was then stained overnight in 1X SYBR Green nucleic acid stain (Molecular Probes) or another stain in order to visualize the pDNA in the gel.

### Example 2

### Size Distribution of Micellar Complexes

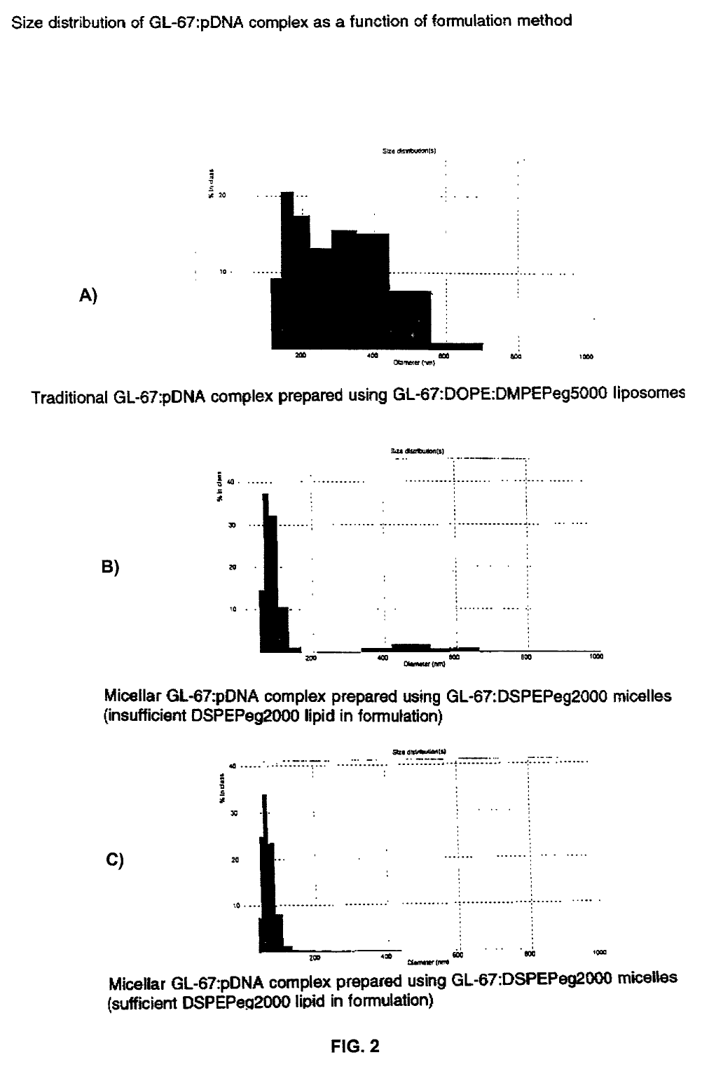

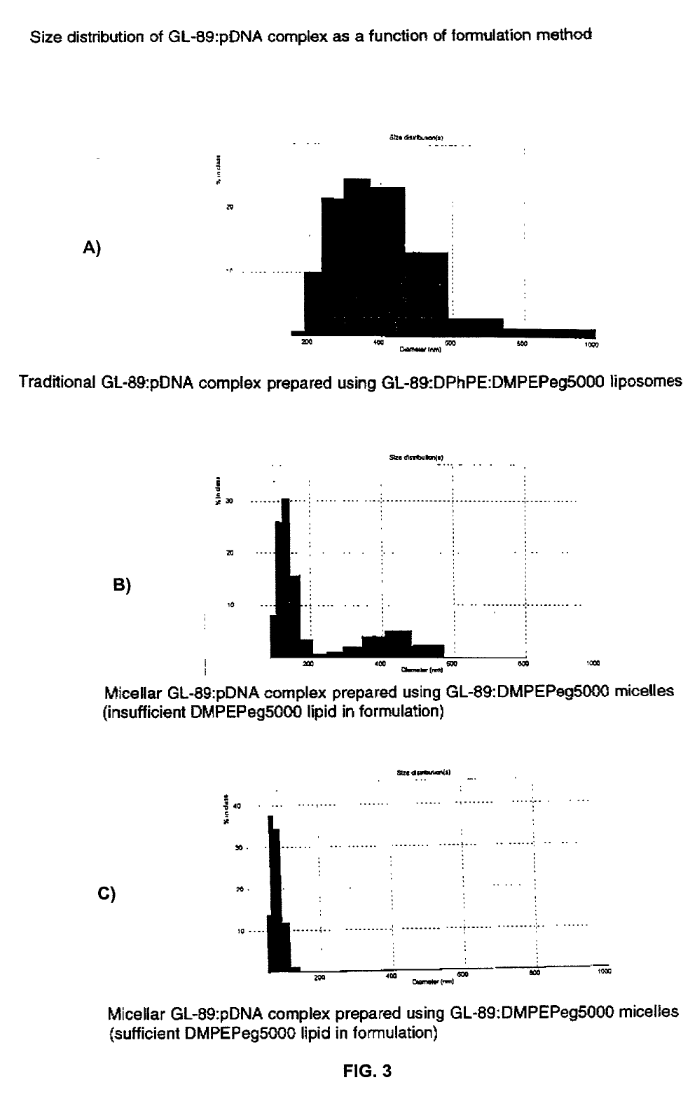

In FIGS. 2 and 3 the size distribution of traditional cationic lipid:pDNA complexes (FIGS. 2A & 3A) are compared to the size distribution of cationic lipid:pDNA complexes prepared via the micellar method (FIGS. 2B, 2C, 3B, & 3C).

The size distribution of a complex was determined by quasi-elastic light scattering with a Malvern Zeta-Sizer 4. The complex was sized within 1 hour of preparation and the complex was measured at the manufacturers recommended count rate of 50-250 kilocounts per second (KCPS). If necessary, the count rate of the sample was adjusted to the desired range of 50-250 KCPS by dilution with water.

For the results depicted in FIG. 2A, a cationic lipid:pDNA complex utilizing cationic lipid GL-67 was prepared in the traditional method. See U.S. Pat. Nos. 5,747,471 and 5,650,096, the disclosures of which are specifically incorporated by reference herein. In brief, the cationic lipid formulation containing the cationic lipid, co-lipid, and the PEG lipid was either 1) dried down to a lipid film from chloroform or 2) lyophilized from t-butanol:water (9:1, vol:vol). The resulting preparation was then hydrated to two times the desired final concentration of the three lipids in the complex using distilled water. The cationic lipid:pDNA complex is prepared by adding an equal volume of lipid to the pDNA followed by gentle mixing. The same procedure was followed replacing GL-67 with GL-89 for the size distributions depicted in FIG. 3A.

In FIGS. 2B and 2C, a cationic lipid:pDNA complex utilizing cationic lipid GL-89 was prepared via the micellar method. First, the micellar cationic lipid:PEG lipid solutions were prepared as follows. GL-89 was hydrated at four times the concentration of the desired final cationic lipid concentration of the cationic lipid:pDNA complex. The PEG containing lipid was also hydrated at four times the concentration of the desired final PEG lipid concentration of the cationic lipid:pDNA complex. Once hydrated, the cationic lipid was added to the PEG lipid at a 1:1 (vol:vol) ratio. The plasmid DNA was then diluted to two times the desired final pDNA concentration of the cationic lipid:pDNA complex. The cationic lipid:PEG lipid:pDNA complex was then prepared by adding the micellar cationic lipid:PEG lipid solution to the PDNA at a 1:1 ratio (vol:vol). The same procedure was followed replacing GL-67 with GL-89 for the size distributions depicted in FIGS. 3B and 3C.

The size distributions of the traditional cationic lipid complexes, as seen in FIGS. 2A and 3A, are quite large varying from 200 nm to 1000 nm, for example, for GL-67. The size distribution of the traditional complexes does not vary significantly as a function of the cationic lipid:pDNA ratio. In FIGS. 2B & 3B a micellar complex is formed, however, a minimum amount of PEG lipid to form the preferred stable, homogeneous micellar complexes is not present. As a result, the size distribution in FIGS. 2B & 3B extends to sizes of greater than 400 nm. Finally, in FIGS. 2C & 3C, the size distribution of the preferred micellar complexes prepared with a sufficient amount of PEG lipid are depicted. The size distribution of the preferred micellar complexes is significantly more homogeneous than traditional cationic lipid complexes and also significantly more homogeneous than micellar lipid complexes lacking an effective amount of PEG lipid.

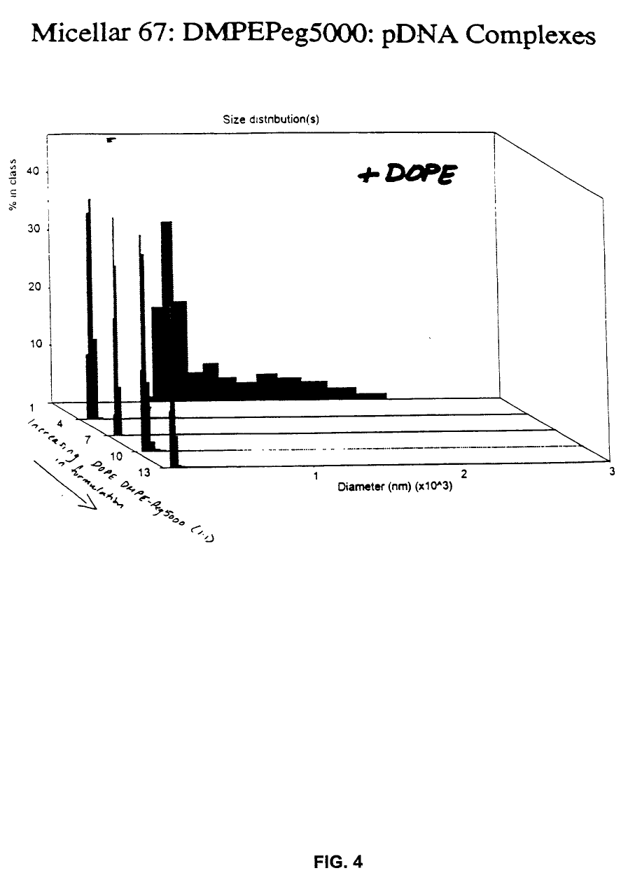

Another example of the difference in size distribution of micellar complexes that are lacking an effective amount of PEG lipid and the preferred micellar complexes of the present invention which contain an effective amount of PEG lipid is shown in FIG. 4. Once an effective amount of PEG lipid is added the size distribution becomes significantly more homogeneous.

### Example 3

### Binding of Traditional and Micellar Cationic Lipid Complexes to Airway Epithelial Cells

The following example examines the ability of both traditional and micellar cationic lipid:pDNA complexes to bind to the surface of polarized normal human bronchial airway cells.

Growth of Polarized Normal Human Epithelial Cells at an Air-liquid Interface

Cell culture flasks were coated with human collagen by dissolving human collagen (Sigma, human placental collagen, #7521) to 50 mg/100 mL in 0.2% glacial acetic acid. Once dissolved, the collagen solution is filtered through a 0.45 μm filter set-up. This concentrated, sterile stock may be stored for 6 months at 4 C. The solution was prepared for coating of the flasks by diluting collagen stock 1:5 (vol:vol) in sterile distilled water several minutes prior to use. The appropriate volume of diluted collagen was placed into a flask (12 mL for T75 flasks, 24 mL for T150 flasks, and 400 μL for each Millicell-PCF insert) and left for at least 2 hrs (preferably overnight) at 4 C. Following incubation at 4 C, collagen was removed from flasks/inserts and left in a sterile hood to dry for 6-12 hours. The flasks/inserts were rinsed twice with sterile phosphate buffered saline, pH 7.4 containing penicillin/streptomycin. These flasks may be kept at room temperature for up to 6 months.

A vial of normal human bronchial epithelial cells (Clonetics) was thawed rapidly and split into five T150 flasks which have been pre-coated with human collagen as indicated above. Cells were grown in the flasks with DMEM (Gibco/BRL):BEGM (Clonetics) 1:1 (vol:vol) media in a 5% CO₂ environment. Cells were grown to 80-90% confluence. Cells were then placed in Millicell-PCF inserts (200 μl@2×10⁵ cells/200 μl media) which were pre-coated with human collagen as indicated above. Twenty four hours after seeding, media was removed from the insert interior and media on the exterior of the insert was replaced with fresh media. Cells were then maintained in the air-liquid interface condition by replacing the exterior media every other day. Approximately 5-7 days following the switch to the air-liquid interface condition, cells developed a high trans-epithelial resistance.

Examination of the Binding of Traditional and Micellar Complexes to the Surface of Polarized Normal Human Bronchial Airway Epithelial Cells

Normal human bronchial airway epithelial cells were grown at an air-liquid interface as described above. Cells were maintained at the air-liquid interface for approximately five days or until a trans-epithelial resistance of approximately 1000 Ω/cm² developed. The cells were then ready for use in the binding experiment.

Plasmid DNA was labeled non-covalently with the fluorescent probe Toto-1-iodide (Molecular Probes) at a 1:200 molar ratio of Toto-1:pDNA according to the manufacturer's instructions. Micellar and traditional lipid:pDNA complexes were prepared at 10 times the desired lipid:pDNA concentration to be used in the experiment using Toto-1 labeled plasmid. The micellar complex was prepared as described above. The traditional complexes were prepared by hydrating the traditional lipid films with water to 20 times the final experimental lipid concentration desired. Labeled pDNA was prepared at 20 times the final experimental DNA concentration desired. The lipid was added to the labeled pDNA and allowed to complex for 15 minutes. The complexes were then diluted 1:10 (vol:vol) in Optimem.

The complexes (approx. 300μl) were added to the apical membranes of the airway epithelial cells in the interior of the insert and allowed to bind for 1 hour at 37 C in a 5% CO₂ atmosphere. The complex was then aspirated from the cell surface; the cell surface was washed 3 times with 0.5 mL cold PBS; fixed for 15 minutes in 2% paraformaldehyde in PBS; and washed once with 0.5 mL cold PBS. The insert was then cut out from the insert housing, placed on a slide, coversliped, and mounted with Immunomount (Shandon-Lipshaw) containing 2 μg/mL DAPI as a nuclear counterstain.

Robust binding of the micellar complexes to the apical surface of the airway cells was generally observed at cationic lipid:pDNA ratios of 50:50, 75:50 μM. There was negligible binding of traditional complexes in the same environment at cationic lipid:pDNA ratios of 50:50, 75:50 μM. This methodology should be applicable to essentially any adherent cell line.

We claim:

1. A method of making micellar complexes comprising:
a) combining at least one cationic lipid with a sufficient amount of PEG derivative in an amount suitable to produce substantially homogeneous micellar lipids;
b) combining said substantially homogeneous micellar lipids and at least one biologically active molecule to form said micellar complexes.

2. A method of making micellar complexes according to claim 1, wherein said PEG derivative is complexed to a co-lipid prior to step a).

3. A method of making micellar complexes according to claim 1, wherein said biologically active molecule is DNA.

4. A method of making micellar complexes according to claim 4, wherein said at least one cationic lipid and said DNA are present in a lipid:DNA ratio of 1:8.

5. A method of making micellar complexes according to claim 1, wherein the size distribution of a group of micellar complexes varies by less than 20% relative to the average size of a complex in said group of micellar complexes.

6. A method of making micellar complexes according to claim 1, further comprising the step of coating said micellar complexes with at least one hydrophobic species.

7. A method of making micellar complexes according to claim 1, further comprising the addition of an agent for targeting a mammalian cell.

8. A method of making micellar complexes according to claim 7, wherein said agent for targeting is selected from peptides containing a RGD, UDP/UTP, lactose, cyclic RGD peptide, penetratin, lectins, agents to target the LDL receptor, mannose-6-phosphate, HAV peptides, CNP-22 peptides and airway specific single chain antibodies.

9. A micellar complex produced according to claim 1.

10. A micellar complex produced according to claim 2.

11. A micellar complex according to claim 9, wherein said micellar complex further comprises an agent for targeting a mammalian cell.

12. A micellar complex according to claim 11, wherein said agent for targeting is selected from peptides containing a RGD, UDP/UTP, lactose, cyclic RGD peptide, penetratin, lectins, agents to target the LDL receptor, mannose-6-phosphate, HAV peptides, CNP-22 peptides and airway specific single chain antibodies.

13. A micellar complex according to claim 9, wherein said micellar complex further comprises a hydrophobic species to coat said micellar complex.

14. A micellar complex according to claim 9, wherein said wherein said biologically active molecule is DNA.

15. A micellar complex according to claim 14, wherein said at least one cationic lipid and said DNA are present in a lipid:DNA ratio of 1:8.

16. A micellar complex according to claim 9, wherein the size distribution of a group of micellar complexes varies by less than 20% relative to the average size of a complex in said group of micellar complexes.

17. A method of delivering a biologically active molecule to a cell of a mammal comprising contacting said cell with a composition comprising a micellar complex, wherein said micellar complex comprises:
at least one cationic lipid;
at least one biologically active molecule; and
a least one PEG derivative.

18. A method of delivering a biologically active molecule to a cell of a mammal according to claim 17, wherein said micellar complex further comprises a co-lipid.

19. A method of delivering a biologically active molecule to a cell of a mammal according to claim 17, wherein said at least one biologically active molecule is DNA.

20. A method of delivering a biologically active molecule to a cell of a mammal according to claim 19, wherein said at least one cationic lipid and said DNA are present in a lipid:DNA ratio of 1:8.

21. A method of delivering a biologically active molecule to a cell of a mammal according to claim 17, wherein said micellar complex further comprises a hydrophobic species to coat said micellar complex.

22. A method of delivering a biologically active molecule to a cell of a mammal according to claim 17, wherein said micellar complex further comprises an agent for targeting a mammalian cell.

23. A method of delivering a biologically active molecule to a cell of a mammal according to claim 22, wherein said agent for targeting is selected from peptides containing a RGD sequence, UDP/UTP, lactose, cyclic RGD peptide, penetratin, lectins, agents to target the LDL receptor, mannose-6-phosphate, HAV peptides, CNP-22 peptides and airway specific single chain antibodies.

24. A method of delivering a biologically active molecule to a cell of a mammal according to claim 17, wherein said cell is an airway epithelial cell.

25. A micellar complex comprising:
at least one cationic lipid:
at least one PEG derivative; and
at least one biologically active molecule;
wherein the size distribution of a group of micellar complexes comprising said micellar complex has a substantially homogeneous size distribution.

26. A micellar complex according to claim 25, wherein said micellar complex further comprises a co-lipid.

27. A micellar complex according to claim 25, wherein said substantially homogeneous size distribution of said group of micellar complexes varies by less than 20% relative to the average size of a complex in said group of micellar complexes.

28. A micellar complex according to claim 25, wherein said biologically active molecule is DNA.

29. A micellar complex according to claim 25, wherein said micellar complex further comprises an agent for targeting a mammalian cell.

30. A micellar complex according to claim 29, wherein said agent for targeting is selected from peptides containing a RGD sequence, UDP/UTP, lactose, cyclic RGD peptide, penetratin, lectins, agents to target the LDL receptor, mannose-6-phosphate, HAV peptides, CNP-22 peptides and airway specific single chain antibodies.
